(19) United States
(12) Patent Application Publication
(10) Pub. No.: US 2023/0270408 A1
(43) Pub. Date: Aug. 31, 2023

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Kenichi Shidara, Kanagawa (JP)

(73) Assignee: FUJIFILM Corporation, Tokyo (JP)

(21) Appl. No.: 18/064,146

(22) Filed: Dec. 9, 2022

(30) Foreign Application Priority Data
Feb. 25, 2022 (JP) 2022-027503

(51) Int. Cl. A61B 8/00; A61B 8/08

(52) U.S. Cl. CPC A61B 8/4477; A61B 8/5246; A61B 8/54

(57) ABSTRACT

To provide an ultrasound diagnostic apparatus and a control method of the ultrasound diagnostic apparatus in which a user can smoothly perform an examination on a subject while simultaneously using a plurality of ultrasound probes.

An ultrasound diagnostic apparatus includes a plurality of ultrasound probes having communication circuits that transmit synchronization signals, and operate according to a time sequence including an ultrasonic wave transmission/reception period for transmitting and receiving ultrasonic waves, a waiting period for stopping transmission and reception of the ultrasonic waves, and a synchronization period for synchronization between the ultrasound probes. In a case where any ultrasound probe is in the ultrasonic wave transmission/reception period, the other ultrasound probe is in the waiting period. In a case where any ultrasound probe is in the synchronization period, the other ultrasound probe is also in the synchronization period. In a case where the synchronization is failed, the synchronization period is extended and after the plurality of ultrasound probes becomes a state capable of synchronizing with each other, any ultrasound probe is in the ultrasonic wave transmission/reception period.

### CROSS-REFERENCE TO RELATED APPLICATIONS

The present application claims priority under 35 U.S.C. § 119 to Japanese Patent Application No. 2022-027503, filed on Feb. 25, 2022. The above application is hereby expressly incorporated by reference, in its entirety, into the present application.

### BACKGROUND OF THE INVENTION

### 1. Field of the Invention

The present invention relates to an ultrasound diagnostic apparatus including a plurality of ultrasound probes and a control method of the ultrasound diagnostic apparatus.

### 2. Description of the Related Art

In the related art, a plurality of portions of a subject are simultaneously examined using a plurality of ultrasound probes. At this time, it is known that in a case where ultrasonic waves are simultaneously transmitted from the plurality of ultrasound probes to the subject, the ultrasonic waves transmitted from the plurality of ultrasound probes interfere with each other, and an image quality of an acquired ultrasound image deteriorates. Therefore, in order to prevent deterioration of the image quality due to interference between the ultrasonic waves, for example, as disclosed in WO2019/145141A, an ultrasound diagnostic apparatus has been developed in which a plurality of ultrasound probes are synchronized to transmit ultrasonic waves to a subject in order from the plurality of ultrasound probes.

### SUMMARY OF THE INVENTION

However, synchronization between the plurality of ultrasound probes may fail for some reason. In this case, ultrasonic waves may not be transmitted to the subject in order from the ultrasound probes between which synchronization has failed, and a user may not be able to smoothly perform the examination on the subject while observing the ultrasound image.

The present invention has been made to solve such a problem in the related art, and has an object to provide an ultrasound diagnostic apparatus and a control method of the ultrasound diagnostic apparatus in which a user can smoothly perform an examination on a subject while simultaneously using a plurality of ultrasound probes.

In order to achieve the above-described object, an ultrasound diagnostic apparatus according to an aspect of the present invention comprises a plurality of ultrasound probes each transmitting and receiving ultrasonic waves, in which the plurality of ultrasound probes have communication circuits that notify of an operating state regarding transmission and reception of the ultrasonic waves by transmitting synchronization signals to each other, each of the plurality of ultrasound probes operates according to a time sequence including at least an ultrasonic wave transmission/reception period for transmitting and receiving the ultrasonic waves, a waiting period for stopping transmission and reception of the ultrasonic waves, and a synchronization period for synchronization between the ultrasound probes, in a case where any one of the plurality of ultrasound probes is in the ultrasonic wave transmission/reception period, the other ultrasound probe is in the waiting period, in a case where any one of the plurality of ultrasound probes is in the synchronization period, the other ultrasound probe is also in the synchronization period, and in a case where the plurality of ultrasound probes fail to synchronize with each other in the synchronization period, the synchronization period is extended and after the plurality of ultrasound probes becomes a state capable of synchronizing with each other, any one of the plurality of ultrasound probes is in the ultrasonic wave transmission/reception period.

The ultrasound diagnostic apparatus may comprise an input device for a user to perform an input operation, and in a case where the synchronization period is extended, the synchronization period may be returned to a length before the extension only in a case where the user operates via the input device.

One ultrasound probe of the plurality of ultrasound probes may operate as a main probe, the remaining ultrasound probe may operate as a subordinate probe, and the main probe may have a synchronization period extension unit for extending the synchronization period in a case where the communication circuit is not capable of detecting the synchronization signal transmitted from the subordinate probe in the synchronization period.

The synchronization period extension unit may automatically extend the synchronization period in a case where the communication circuit is not capable of detecting the synchronization signal transmitted from the subordinate probe in the synchronization period.

In addition, the synchronization period extension unit may extend the synchronization period based on the input operation by the user via the input device.

A maximum value of the extension of the synchronization period may be defined.

The synchronization period may be extended in a stepwise manner.

The ultrasound diagnostic apparatus may comprise a plurality of apparatus main bodies that correspond to the plurality of ultrasound probes and are connected to the plurality of ultrasound probes, respectively, and each of the plurality of ultrasound probes may acquire image data by transmitting and receiving the ultrasonic waves in the ultrasonic wave transmission/reception period and transmit the image data to the corresponding apparatus main body.

Each of the plurality of ultrasound probes may wirelessly transmit the synchronization signal to the other ultrasound probe, and each of the plurality of ultrasound probes may transmit the image data to the corresponding apparatus main body by wire.

Each of the plurality of ultrasound probes may transmit the synchronization signal to the other ultrasound probe by a first wireless system, and each of the plurality of ultrasound probes may transmit the image data to the corresponding apparatus main body by a second wireless system.

The plurality of ultrasound probes may transmit the synchronization signal to each other via the plurality of apparatus main bodies.

The ultrasound diagnostic apparatus may comprise one display device connected to the plurality of apparatus main bodies, and a plurality of ultrasound images may be simultaneously displayed on the display device based on a plurality of image data transmitted from the plurality of ultrasound probes to the plurality of apparatus main bodies.

At least one apparatus main body among the plurality of apparatus main bodies may generate a three-dimensional ultrasound image based on the image data transmitted from each of the plurality of ultrasound probes, and the three-dimensional ultrasound image may be displayed on the display device.

Each of the plurality of ultrasound probes may include a transducer array, a transmission/reception circuit that transmits ultrasonic waves from the transducer array and generates a sound ray signal based on a reception signal acquired by the transducer array, and an image data generation unit that generates the image data based on the sound ray signal generated by the transmission/reception circuit.

A control method of an ultrasound diagnostic apparatus according to another aspect of the present invention is a control method of an ultrasound diagnostic apparatus including a plurality of ultrasound probes each transmitting and receiving ultrasonic waves, the control method comprising: by the plurality of ultrasound probes, notifying of an operating state regarding transmission and reception of the ultrasonic waves by transmitting synchronization signals to each other, in which each of the plurality of ultrasound probes operates according to a time sequence including at least an ultrasonic wave transmission/reception period for transmitting and receiving the ultrasonic waves, a waiting period for stopping transmission and reception of the ultrasonic waves, and a synchronization period for synchronization between the ultrasound probes, in a case where any one of the plurality of ultrasound probes is in the ultrasonic wave transmission/reception period, the other ultrasound probe is in the waiting period, in a case where any one of the plurality of ultrasound probes is in the synchronization period, the other ultrasound probe is also in the synchronization period, and in a case where the plurality of ultrasound probes fail to synchronize with each other in the synchronization period, the synchronization period is extended and after the plurality of ultrasound probes becomes a state capable of synchronizing with each other, any one of the plurality of ultrasound probes is in the ultrasonic wave transmission/reception period.

According to the aspects of the present invention, since the ultrasound diagnostic apparatus comprises a plurality of ultrasound probes each transmitting and receiving ultrasonic waves, in which the plurality of ultrasound probes have communication circuits that notify of an operating state regarding transmission and reception of the ultrasonic waves by transmitting synchronization signals to each other, each of the plurality of ultrasound probes operates according to a time sequence including at least an ultrasonic wave transmission/reception period for transmitting and receiving the ultrasonic waves, a waiting period for stopping transmission and reception of the ultrasonic waves, and a synchronization period for synchronization between the ultrasound probes, in a case where any one of the plurality of ultrasound probes is in the ultrasonic wave transmission/reception period, the other ultrasound probe is in the waiting period, in a case where any one of the plurality of ultrasound probes is in the synchronization period, the other ultrasound probe is also in the synchronization period, and in a case where the plurality of ultrasound probes fail to synchronize with each other in the synchronization period, the synchronization period is extended and after the plurality of ultrasound probes becomes a state capable of synchronizing with each other, any one of the plurality of ultrasound probes is in the ultrasonic wave transmission/reception period, the user can smoothly perform the examination on the subject while simultaneously using the plurality of ultrasound probes.

### BRIEF DESCRIPTION OF THE DRAWINGS

FIG. 1 is a block diagram showing a configuration of an ultrasound diagnostic apparatus according to a first embodiment of the present invention.

FIG. 2 is a block diagram showing a configuration of a transmission/reception circuit in the first embodiment of the present invention.

FIG. 3 is a block diagram showing a configuration of an image data generation unit in the first embodiment of the present invention.

FIG. 4 is a diagram schematically showing an example of a time sequence of basic operations of a main probe and a subordinate probe in the first embodiment of the present invention.

FIG. 5 is a diagram schematically showing an example of a time sequence of operations of the main probe and the subordinate probe in a case where synchronization fails in the first embodiment of the present invention.

FIG. 6 is a diagram schematically showing an example of a synchronization period extended once in the first embodiment of the present invention.

FIG. 7 is a diagram schematically showing an example of a synchronization period extended twice in the first embodiment of the present invention.

FIG. 8 is a flowchart showing an operation of the ultrasound diagnostic apparatus according to the first embodiment of the present invention.

FIG. 9 is a diagram schematically showing an example of a time sequence of basic operations of a main probe, a first subordinate probe, and a second subordinate probe in a modification of the first embodiment of the present invention.

FIG. 10 is a diagram schematically showing an example of a time sequence of operations of the main probe, the first subordinate probe, and the second subordinate probe in the modification of the first embodiment of the present invention in a case where the synchronization fails.

FIG. 11 is a block diagram showing a configuration of an ultrasound diagnostic apparatus according to a second embodiment of the present invention.

### DESCRIPTION OF THE PREFERRED EMBODIMENTS

Hereinafter, embodiments of the present invention will be described with reference to the accompanying drawings.

The explanation of configuration requirements described below is based on a representative embodiment of the present invention, but the present invention is not limited to such an embodiment.

In addition, in the present specification, numerical ranges represented by using “˜” mean ranges including numerical values described before and after “18 ” as a lower limit value and an upper limit value.

In the present specification, “same” includes an error range generally allowed in the technical field.

First Embodiment

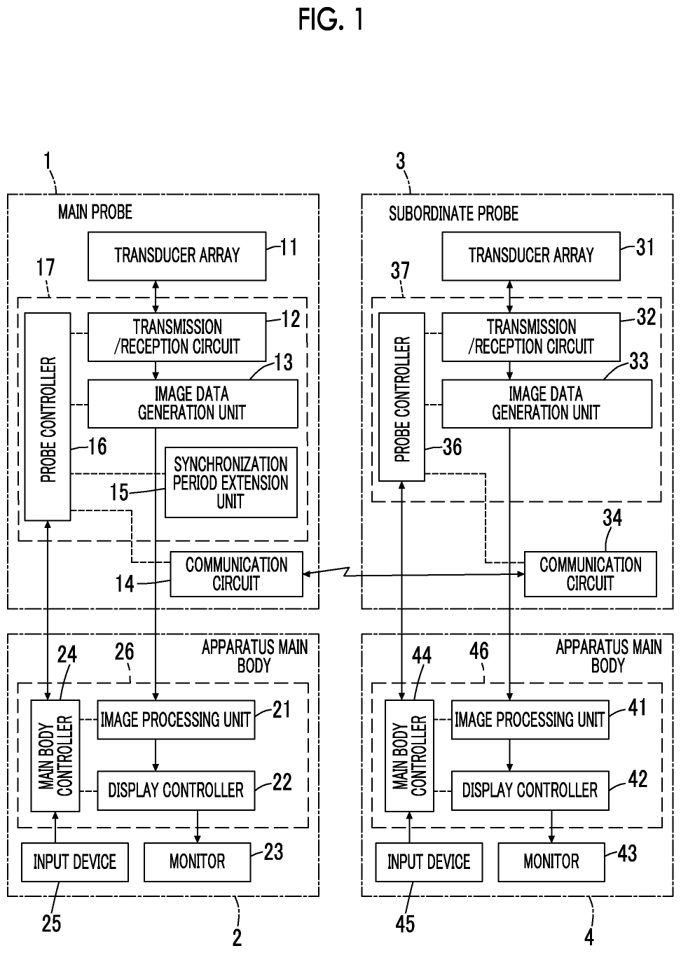

FIG. 1 shows a configuration of an ultrasound diagnostic apparatus according to a first embodiment of the present invention. The ultrasound diagnostic apparatus comprises two ultrasound probes including a main probe 1 and a subordinate probe 3, an apparatus main body 2 connected to the main probe 1, and an apparatus main body 4 connected to the subordinate probe 3. The ultrasound diagnostic apparatus is used, for example, to perform an ultrasound examination of two portions at the same time on the same subject.

The main probe 1 comprises a transducer array 11, and a transmission/reception circuit 12 and an image data generation unit 13 are sequentially connected to the transducer array 11. In addition, the main probe 1 comprises a communication circuit 14 and a synchronization period extension unit 15. Further, a probe controller 16 is connected to the transmission/reception circuit 12, the image data generation unit 13, the communication circuit 14, and the synchronization period extension unit 15.

In addition, the transmission/reception circuit 12, the image data generation unit 13, the synchronization period extension unit 15, and the probe controller 16 constitute a processor 17 for the main probe 1.

The apparatus main body 2 connected to the main probe 1 comprises an image processing unit 21 connected to the image data generation unit 13 of the main probe 1. A display controller 22 and a monitor 23 are sequentially connected to the image processing unit 21. In addition, a main body controller 24 is connected to the image processing unit 21 and the display controller 22. The main body controller 24 is connected to the probe controller 16 of the main probe 1. Further, an input device 25 is connected to the main body controller 24.

In addition, the image processing unit 21, the display controller 22, and the main body controller 24 constitute a processor 26 for the apparatus main body 2.

The subordinate probe 3 comprises a transducer array 31, and a transmission/reception circuit 32 and an image data generation unit 33 are sequentially connected to the transducer array 31. Further, the subordinate probe 3 comprises a communication circuit 34 that communicates with the communication circuit 14 of the main probe 1. Further, a probe controller 36 is connected to the transmission/reception circuit 32, the image data generation unit 33, and the communication circuit 34.

In addition, the transmission/reception circuit 32, the image data generation unit 33, and the probe controller 36 constitute a processor 37 for the subordinate probe 3.

The apparatus main body 4 connected to the subordinate probe 3 comprises an image processing unit 41 connected to the image data generation unit 33 of the subordinate probe 3. A display controller 42 and a monitor 43 are sequentially connected to the image processing unit 41. Further, the main body controller 44 is connected to the image processing unit 41 and the display controller 42. A main body controller 44 is connected to the probe controller 36 of the subordinate probe 3. Further, an input device 45 is connected to the main body controller 44.

In addition, the image processing unit 41, the display controller 42, and the main body controller 44 constitute a processor 46 for the apparatus main body 4.

The transducer array 11 of the main probe 1 has a plurality of ultrasound transducers arranged one-dimensionally or two-dimensionally. Each of the ultrasound transducers transmits ultrasonic waves in accordance with a drive signal supplied from the transmission/reception circuit 12, receives an ultrasound echo from the subject, and outputs a signal based on the ultrasound echo. Each ultrasound transducer is constituted by forming electrodes at both ends of a piezoelectric body composed of, for example, a piezoelectric ceramic represented by lead zirconate titanate (PZT), a polymeric piezoelectric element represented by polyvinylidene difluoride (PVDF), and a piezoelectric single crystal represented by lead magnesium niobate-lead titanate (PMN-PT).

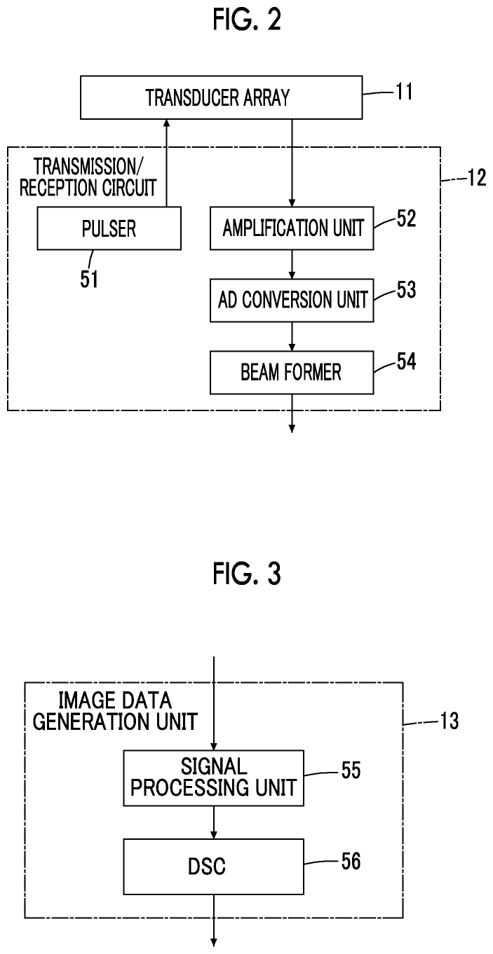

Under control of the probe controller 16, the transmission/reception circuit 12 transmits ultrasonic waves from the transducer array 11 and generates a sound ray signal based on a reception signal acquired by the transducer array 11. As shown in FIG. 2, the transmission/reception circuit 12 includes a pulser 51 connected to the transducer array 11, and an amplification unit 52, and an AD (Analog to Digital) conversion unit 53, and a beam former 54 that are sequentially connected in series from the transducer array 11.

The pulser 51 includes, for example, a plurality of pulse generators, and supplies respective drive signals to a plurality of ultrasound transducers of the transducer array 11 by adjusting a delay amount such that ultrasonic waves transmitted from the plurality of ultrasound transducers form an ultrasonic beam based on a transmission delay pattern selected in response to a control signal from the probe controller 16. In this way, in a case where a pulsed or continuous-wave voltage is applied to the electrodes of the ultrasound transducers of the transducer array 11, the piezoelectric body expands and contracts, pulsed or continuous-wave ultrasonic waves are generated from the respective ultrasound transducers, and the ultrasonic beam is formed from a composite wave of the ultrasonic waves.

The transmitted ultrasonic beam is reflected by, for example, a target such as a portion of a subject and propagates toward the transducer array 11 of the main probe 1. The ultrasound echo propagating toward the transducer array 11 in this way is received by each of the ultrasound transducers constituting the transducer array 11. At this time, each of the ultrasound transducers constituting the transducer array 11 expands and contracts by receiving the propagating ultrasound echo to generate reception signals which are electric signals, and outputs the reception signals to the amplification unit 52.

The amplification unit 52 amplifies the signals input from each of the ultrasound transducers constituting the transducer array 11, and transmits the amplified signals to the AD conversion unit 53. The AD conversion unit 53 converts the signals transmitted from the amplification unit 52 into digital reception data. The beam former 54 performs so-called reception focus processing by adding respective reception data received from the AD conversion unit 53 while applying respective delays. By this reception focus processing, the respective reception data converted by the AD conversion unit 53 are phase-adjusted and added, and a sound ray signal in which the focus of the ultrasound echo is narrowed down is acquired.

As shown in FIG. 3, the image data generation unit 33 has a configuration in which a signal processing unit 55 and a digital scan converter (DSC) 56 are connected in series.

The signal processing unit 55 generates a B-mode image signal which is tomographic image information about a tissue in the subject by performing correction for attenuation by a distance according to a depth of a reflection position of the ultrasonic wave by using a sound velocity value set by the probe controller 16 and then performing envelope detection processing, on the sound ray signal received from the transmission/reception circuit 12.

The DSC 56 converts (raster-converts) the B-mode image signal generated by the signal processing unit 55 into an image signal according to a normal television signal scanning method. The image signal thus obtained is called image data.

The communication circuit 14 and the communication circuit 34 of the subordinate probe 3 transmits the synchronization signal to each other by so-called wireless communication or so-called wired communication, thereby notifying each other of the operation related to transmission and reception of the ultrasonic wave. The communication circuit 14 has, for example, an antenna for transmitting and receiving radio waves, a terminal for connecting a communication cable, and the like. As a communication system for wireless communication, for example, Bluetooth (registered trademark) can be used, and Wi-Fi (registered trademark) or an ultra-wide band (UWB) can also be used.

The probe controller 16 controls each unit of the main probe 1 according to a program recorded in advance or the like. Further, the probe controller 16 performs control such that the main probe 1 operates according to a time sequence including a transmission/reception condition setting period for setting a transmission/reception condition of the ultrasonic waves by performing communication between the main probe 1 and the subordinate probe 3, an ultrasonic wave transmission/reception period for transmitting and receiving the ultrasonic waves, a waiting period for stopping transmission and reception of the ultrasonic waves, and a synchronization period for synchronization with the subordinate probe 3.

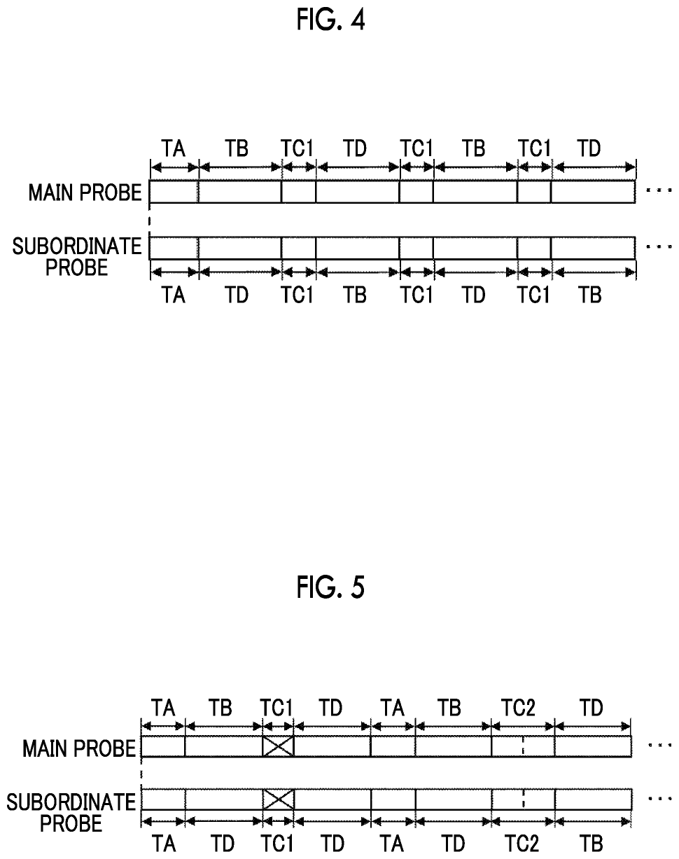

For example, as shown in FIG. 4, the time sequence of the operation of the main probe 1 can be set such that after the transmission/reception condition setting period TA, the ultrasonic wave transmission/reception period TB, the synchronization period TC1, and the waiting period TD are sequentially repeated. At this time, the time sequence of the operation of the subordinate probe 3 is set such that the ultrasonic wave transmission/reception period TB of the main probe 1 and the ultrasonic wave transmission/reception period TB of the subordinate probe 3 do not overlap each other. For example, the time sequence of the operation of the subordinate probe 3 can be set such that after the transmission/reception condition setting period TA, the waiting period TD, the synchronization period TC1, and the ultrasonic wave transmission/reception period TB are sequentially repeated. Here, the ultrasonic wave transmission/reception period TB and the waiting period TD are periods of the same length, and can be set to, for example, about 80 milliseconds. Further, the synchronization period TC1 can be set to, for example, several milliseconds.

Further, in the synchronization period TC1, the probe controller 16 generates a synchronization signal for synchronization with the subordinate probe 3, and transmits the generated synchronization signal to the subordinate probe 3 via the communication circuit 14, thereby performing processing for synchronizing the operation of the main probe 1 with the operation of the subordinate probe 3 such that the end of the synchronization period TC1 of the main probe 1 and the end of the synchronization period TC1 of the subordinate probe 3 are matched. In addition, the probe controller 16 detects a state of the subordinate probe 3 in the time sequence, that is, detects whether the subordinate probe 3 is in the ultrasonic wave transmission/reception period TB, in the synchronization period TC1, or in the waiting period TD. At this time, for example, the probe controller 16 can detect the state of the subordinate probe 3 by receiving a synchronization signal from the subordinate probe 3 in response to the synchronization signal transmitted to the subordinate probe 3.

In addition, in a case where synchronization between the main probe 1 and the subordinate probe 3 fails in the synchronization period TC1, the probe controller 16 returns the state of the main probe 1 to the transmission/reception condition setting period TA as shown in FIG. 5 so that the main probe 1 can be synchronized with the subordinate probe 3, and then operates the main probe 1 again in a predetermined order such as the ultrasonic wave transmission/reception period TB, the synchronization period TC1, the waiting period TD, and the like according to the time sequence.

Here, in a case where the probe controller 16 cannot detect the state of the subordinate probe 3 over the synchronization period TC 1, that is, communication between the main probe 1 and the subordinate probe 3 cannot performed, and it is not possible to determine whether or not the synchronization is normally performed, the probe controller 16 can determine that synchronization between the main probe 1 and the subordinate probe 3 has failed in the synchronization period TC1. For example, in a case where the communication circuit 14 of the main probe 1 is connected to the subordinate probe 3 by wireless communication, it is considered that radio transmission of the synchronization signal is disturbed by electromagnetic noise from the outside, so that the probe controller 16 cannot detect the state of the subordinate probe 3 over the synchronization period TC1. The electromagnetic noise from the outside includes, for example, communication signals emitted from a communication device such as a wireless local area network (LAN) device or a Bluetooth (registered trademark) device, electromagnetic waves emitted from an electric device such as an electrosurgical knife, a radiation device, or an air purifier, and the like.

Further, the probe controller 16 can determine that synchronization between the main probe 1 and the subordinate probe 3 is successful in a case where the probe controller 16 can detect the state of the subordinate probe 3 in the synchronization period TC1.

In addition, the transmission/reception conditions of the ultrasonic waves include an order of the ultrasonic wave transmission/reception period TB, the synchronization period Cl, and the waiting period TD, the number of frames of the ultrasound image acquired in the ultrasonic wave transmission/reception period TB, and the like in the main probe 1 and the subordinate probe 3.

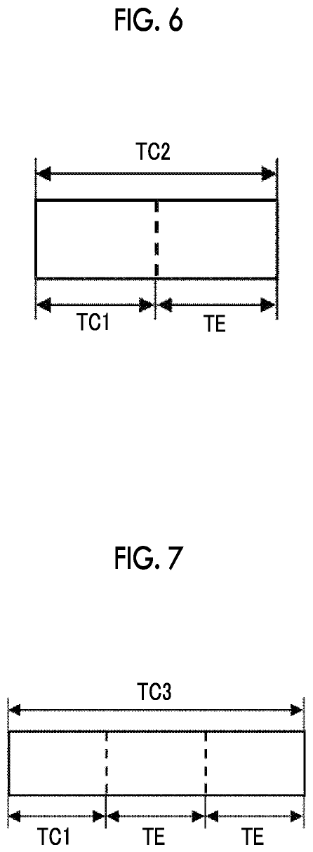

For example, as shown in FIG. 5, in a case where synchronization between the main probe 1 and the subordinate probe 3 fails in the synchronization period TC1, the synchronization period extension unit 15 extends the synchronization period TC1 and sets a new synchronization period TC2. For example, as schematically shown in FIG. 6, the synchronization period extension unit 15 stores a predetermined extension period TE in advance, and can set the synchronization period TC2 by adding the extension period TE to the synchronization period TC1. The extension period TE can be set to the same length as that of the synchronization period TC1.

In addition, the synchronization period extension unit 15 can further extend the synchronization period TC1 each time synchronization between the main probe 1 and the subordinate probe 3 fails. For example, in a case where synchronization between the main probe 1 and the subordinate probe 3 fails in the synchronization period TC2 after the synchronization period TC1 is extended and the synchronization period TC2 is set, the synchronization period extension unit 15 can set the synchronization period TC3 schematically shown in FIG. 7 by adding the extension period TE to the synchronization period TC2.

Further, the processor 17 including the transmission/reception circuit 12, the image data generation unit 13, the synchronization period extension unit 15, and the probe controller 16 of the main probe 1 is configured of a central processing unit (CPU) and a control program for causing the CPU to perform various types of processing, but may be configured using a field programmable gate array (FPGA), a digital signal processor (DSP), an application specific integrated circuit (ASIC), a graphics processing unit (GPU), or other integrated circuit (IC), or may be configured of a combination thereof.

In addition, the transmission/reception circuit 12, the image data generation unit 13, the synchronization period extension unit 15, and the probe controller 16 of the processor 17 can also be configured by being partially or wholly integrated into one CPU or the like.

The image processing unit 21 of the apparatus main body 2 performs various types of necessary image processing such as gradation processing on the image data input from the image data generation unit 13 of the main probe 1, and then sends the image data to the display controller 22. Hereinafter, the image data subjected to various types of necessary image processing such as gradation processing is referred to as an ultrasound image.

Under the control of the main body controller 24, the display controller 22 performs predetermined processing on the ultrasound image or the like generated by the image processing unit 21 and displays the ultrasound image or the like on the monitor 23.

The input device 25 is used for the user to perform an input operation. The input device 25 is configured of a device used for the user to perform an input operation, for example, a keyboard, a mouse, a track ball, a touch pad, a touch panel and the like.

The main body controller 24 controls each unit of the apparatus main body 2 according to a program recorded in advance or the like. At this time, the main body controller 24 can control each unit of the apparatus main body 2 so as to receive information on the time sequence of the main probe 1 from the probe controller 16 of the main probe 1, and to perform processing of the image data and display of the ultrasound image based on the received time sequence information. In addition, the main body controller 24 can control each unit of the apparatus main body 2 based on the instruction information input from the user via the input device 25. In addition, the main body controller 24 can transmit instruction information input from the user to the probe controller 16 of the main probe 1. At this time, the probe controller 16 of the main probe 1 can control each unit of the main probe 1 so as to transmit and stop the ultrasonic waves based on the received instruction information.

Further, the processor 26 including the image processing unit 21, the display controller 22, and the main body controller 24 of the apparatus main body 2 is configured of a CPU and a control program for causing the CPU to perform various types of processing, but may be configured using an FPGA, a DSP, an ASIC, a GPU, or other ICs, or may be configured of a combination thereof.

In addition, the image processing unit 21, the display controller 22, and the main body controller 24 of the processor 26 can also be configured by being partially or wholly integrated into one CPU or the like.

Since the transducer array 31, the transmission/reception circuit 32, the image data generation unit 33, the communication circuit 34, and the probe controller 36 of the subordinate probe 3 are the same as the transducer array 11, the transmission/reception circuit 12, the image data generation unit 13, the communication circuit 14 and the probe controller 16 of the main probe 1, respectively, detailed descriptions on the transducer array 31, the transmission/reception circuit 32, the image data generation unit 33, the communication circuit 34, and the probe controller 36 of the subordinate probe 3 will be omitted.

Further, the processor 37 including the transmission/reception circuit 32, the image data generation unit 33, and the probe controller 36 of the subordinate probe 3 is configured of a CPU and a control program for causing the CPU to perform various types of processing, but may be configured using an FPGA, a DSP, an ASIC, a GPU, or other ICs, or may be configured of a combination thereof. In addition, the transmission/reception circuit 32, the image data generation unit 33, and the probe controller 36 of the processor 37 may be configured by being partially or wholly integrated into one CPU or the like.

Since the apparatus main body 4 connected to the subordinate probe 3 is the same as the apparatus main body 2 connected to the main probe 1, a detailed description on the apparatus main body 4 will be omitted.

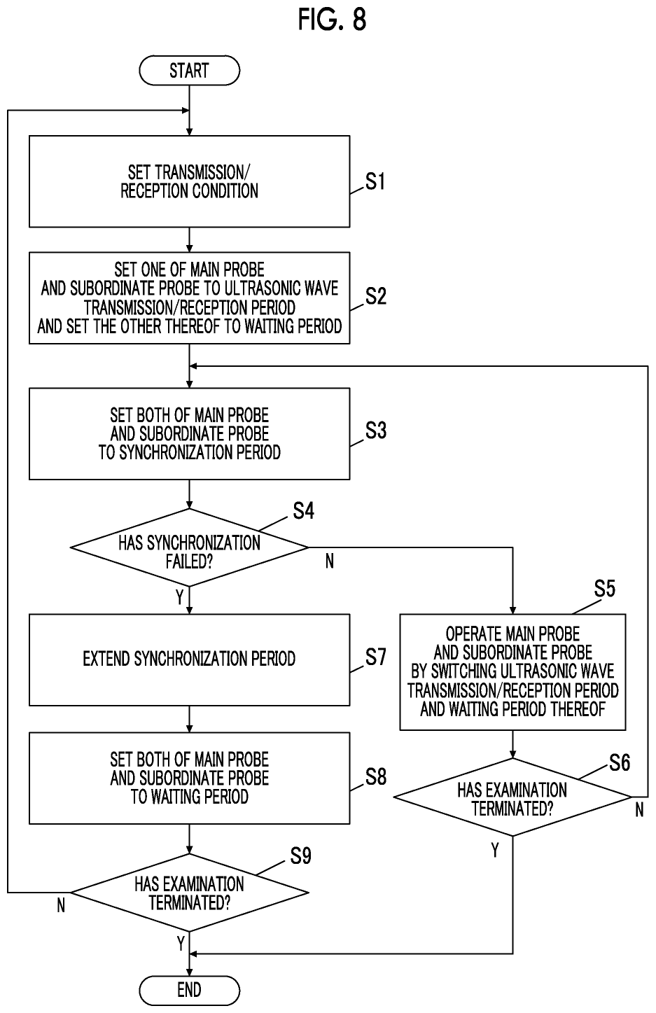

Next, an example of operation of the ultrasound diagnostic apparatus according to the first embodiment will be described with reference to the flowchart of FIG. 8. It is assumed that the main probe 1 and the subordinate probe 3 are in contact with a body surface of the subject in order to capture an ultrasound image representing a tomographic image in the subject.

First, in step S1, as shown in FIG. 4, the probe controller 16 of the main probe 1 and the probe controller 36 of the subordinate probe 3 set the main probe 1 and the subordinate probe 3 to the transmission/reception condition setting period TA, and set transmission/reception conditions of the main probe 1 and the subordinate probe 3 by causing the main probe 1 and the subordinate probe 3 to communicate with each other.

Next, in step S2, the probe controller 16 of the main probe 1 and the probe controller 36 of the subordinate probe 3 set one of the main probe 1 and the subordinate probe 3 to the ultrasonic wave transmission/reception period TB and set the other thereof to the waiting period TD. In the example shown in FIG. 4, the main probe 1 is set to the ultrasonic wave transmission/reception period TB, and the subordinate probe 3 is set to the waiting period TD.

At this time, the ultrasonic waves are transmitted from the transducer array 11 of the main probe 1 to the subject. The transmission/reception circuit 12 performs reception focus processing under the control of the probe controller 16 to generate a sound ray signal. The sound ray signal generated by the transmission/reception circuit 12 in this way is transmitted to the image data generation unit 13. The image data generation unit 13 generates image data by using the sound ray signal transmitted from the transmission/reception circuit 12. The image processing unit 21 processes the image data generated by the image data generation unit 13 to generate an ultrasound image. The ultrasound image generated in this way is sent to the monitor 23 via the display controller 22 and displayed.

In this way, in the ultrasonic wave transmission/reception period TB, ultrasound images are continuously generated by the main probe 1 and the apparatus main body 2, and the ultrasound images are sequentially displayed on the monitor 23. In addition, the subordinate probe 3 stops transmitting the ultrasonic waves to the subject in the waiting period TD which is a period of the same length as the ultrasonic wave transmission/reception period TB. Since the ultrasound image is never generated in the subordinate probe 3, the ultrasound image is not displayed on the monitor 43 of the apparatus main body 4.

Here, in general, in a case where ultrasonic waves are transmitted from a plurality of ultrasound probes to a subject and an ultrasound image of each of examination locations in contact with the plurality of ultrasound probes is captured, the ultrasonic waves emitted from the plurality of ultrasound probes may interfere with each other to cause the image quality to deteriorate, such as inclusion of an artifact in the obtained ultrasound image.

In step S2, since only one of the main probe 1 and the subordinate probe 3 is set to the ultrasonic wave transmission/reception period TB, the ultrasonic waves transmitted from the main probe 1 to the subject and the ultrasonic waves transmitted from the subordinate probe 3 to the subject do not interfere with each other, and deterioration of the image quality of the ultrasound image can be prevented.

In a case where the ultrasonic wave transmission/reception period TB of the main probe 1 and the waiting period TD of the subordinate probe 3 in step S2 have ended, the probe controller 16 of the main probe 1 and the probe controller 36 of the subordinate probe 3 set the main probe 1 and the subordinate probe 3 to the synchronization period TC1 together. In the synchronization period TC1, the probe controller 16 of the main probe 1 and the probe controller 36 of the subordinate probe 3 perform processing for synchronizing the main probe 1 and the subordinate probe 3.

At this time, the probe controller 16 of the main probe 1 generates a synchronization signal and transmits the synchronization signal to the subordinate probe 3 via the communication circuit 14. The communication circuit 34 of the subordinate probe 3 receives the synchronization signal transmitted from the main probe 1 and transmits the synchronization signal to the probe controller 36. Upon receiving the synchronization signal, the probe controller 36 controls each unit of the subordinate probe 3 such that, for example, the synchronization period TC1 of the subordinate probe 3 is ended in conformity with the end of the synchronization period TC1 of the main probe 1. In addition, the probe controller 36 generates a synchronization signal including information indicating that the subordinate probe 3 is in a state of the ultrasonic wave transmission/reception period TB, the synchronization period TC1, or the waiting period TD, and transmits the synchronization signal to the main probe 1 via the communication circuit 34.

In step S4, the probe controller 16 of the main probe 1 determines whether or not synchronization between the main probe 1 and the subordinate probe 3 has failed in the synchronization period TC1 of step S3. The probe controller 16 of the main probe 1 determines that the synchronization is successful, for example, in a case where a synchronization signal is detected from the subordinate probe 3 via the communication circuit 14 in the synchronization period TC1. In addition, the probe controller 16 determines that the synchronization has failed, for example, in a case where the communication state between the main probe 1 and the subordinate probe 3 deteriorates for some reason so that it is not possible to detect the synchronization signal over the synchronization period TC1, and it cannot be determined whether or not the synchronization is normally performed.

In a case where it is determined that the synchronization is successful in step S4, the process proceeds to step S5.

In step S5, the probe controller 16 of the main probe 1 and the probe controller 36 of the subordinate probe 3 operate the main probe 1 and the subordinate probe 3 by switching the ultrasonic wave transmission/reception period TB and the waiting period TD of the main probe 1 and the subordinate probe 3 in step S2. For example, as shown in FIG. 4, in a case where the main probe 1 is set to the ultrasonic wave transmission/reception period TB and the subordinate probe 3 is set to the waiting period TD in step S2, the main probe 1 is set to the waiting period TD, and the subordinate probe 3 is set to the ultrasonic wave transmission/reception period TB in step S5.

In this case, on the monitor 23 of the apparatus main body 2 connected to the main probe 1, so-called freeze display of the ultrasound image is performed in which the latest ultrasound image obtained in step S2 is displayed as a still image. Further, on the monitor 43 of the apparatus main body 4 connected to the subordinate probe 3, the ultrasound images continuously generated by the image processing unit 41 are sequentially displayed.

In a case where the waiting period TD of the main probe 1 and the ultrasonic wave transmission/reception period TB of the subordinate probe 3 have ended, the process proceeds to step S6. In step S6, the probe controller 16 of the main probe 1 and the probe controller 36 of the subordinate probe 3 determine whether or not to terminate the examination for the subject. The probe controller 16 of the main probe 1 and the probe controller 36 of the subordinate probe 3 determine to terminate the examination for the subject in a case where, for example, an instruction for terminating the examination is input by the user via the input device 25 of the apparatus main body 2 and the input device 45 of the apparatus main body 4. In addition, the probe controller 16 of the main probe 1 and the probe controller 36 of the subordinate probe 3 determine to continue the examination in a case where, for example, an instruction for terminating the examination is not input by the user via the input device 25 of the apparatus main body 2 and the input device 45 of the apparatus main body 4.

In a case where it is determined to terminate the examination in step S6, the operation of the ultrasound diagnostic apparatus according to the flowchart of FIG. 8 is terminated.

In a case where it is determined to continue the examination in step S6, the process returns to step S3 and both the main probe 1 and the subordinate probe 3 are set to the synchronization period TC1.

In this way, as long as it is determined that the synchronization between the main probe 1 and the subordinate probe 3 in the synchronization period TC1 is successful in step S4 and it is determined to continue the examination in step S6, the processes of steps S3 to S6 are repeated.

In a case where it is determined that the synchronization has failed in step S4, the process proceeds to step S7.

In step S7, the synchronization period extension unit 15 extends the synchronization period TC1. At this time, for example, as schematically shown in FIG. 6, the synchronization period extension unit 15 can set a new synchronization period TC2 by adding a predetermined extension period TE to the synchronization period TC1. By extending the synchronization period TC1 in this way, it is possible to normally perform synchronization within a period in which there is no electromagnetic noise from the outside and a communication status is stable.

In addition, by extending the synchronization period TC1, a frame rate of the ultrasound image captured by the main probe 1 and a frame rate of the ultrasound image captured by the subordinate probe 3 decrease, but since the certainty of synchronization is improved, for example, it is possible to prevent the user from being unable to perform the examination smoothly due to the failure of synchronization between the main probe 1 and the subordinate probe 3. That is, by extending the synchronization period TC1, the ultrasound image can be stably acquired and displayed on the monitors 23 and 43, and the user can smoothly perform the examination.

In subsequent step S8, the probe controller 16 of the main probe 1 and the probe controller 36 of the subordinate probe 3 set both the main probe 1 and the subordinate probe 3 to the waiting period TD. At this time, for example, the probe controller 16 of the main probe 1 can generate information indicating that the synchronization has failed, and can transmit the information to the subordinate probe 3 via the communication circuit 14. The probe controller 36 of the subordinate probe 3 can control each unit of the subordinate probe 3 such that the subordinate probe 3 is set to the waiting period TD based on the information indicating that the synchronization has failed, which is received by the communication circuit 34.

In step S8, in a case where the waiting period TD of the main probe 1 and the subordinate probe 3 has ended, the process proceeds to step S9. In step S9, the probe controller 16 of the main probe 1 and the probe controller 36 of the subordinate probe 3 determine whether or not to terminate the examination for the subject in the same manner as in step S6. In a case where it is determined to continue the examination in step S9, the process returns to step S1. As a result, the states of the main probe 1 and the subordinate probe 3 return to the transmission/reception condition setting period TA and the main probe 1 and the subordinate probe 3 are in a state where they can be synchronized with each other.

In this way, as long as it is determined that the synchronization has failed in step S4 and it is determined to continue the examination in step S9, the processes of steps S1 to S4 and steps S7 to S9 are repeated.

Here, in step S7, each time the synchronization between the main probe 1 and the subordinate probe 3 fails in step S4, the synchronization period extension unit 15 can further extend the already extended synchronization period. For example, as shown in FIG. 7, in a case where it is determined that the synchronization has failed twice in step S4, a new synchronization period TC3 can be set by adding the extension period TE to the synchronization period TC2, which is a period in which the synchronization period TC1 is extended once.

By extending the synchronization period TC1 in this way, the main probe 1 and the subordinate probe 3 can more reliably perform normal synchronization.

In a case where it is determined to terminate the examination in step S9, the operation of the ultrasound diagnostic apparatus according to the flowchart of FIG. 8 is terminated.

As described above, according to the ultrasound diagnostic apparatus according to the first embodiment of the present invention, in a case where the synchronization between the main probe 1 and the subordinate probe 3 has failed in the synchronization period TC1, the synchronization period extension unit 15 extends the synchronization period TC1. Therefore, the main probe 1 and the subordinate probe 3 can be more reliably synchronized with each other, and the user can smoothly perform the examination on the subject.

Although it has been described that the image processing unit 21 is provided in the apparatus main body 2, the image processing unit 21 may be provided in the main probe 1 instead of the apparatus main body 2. Similarly, the image processing unit 41 may be provided in the subordinate probe 3 instead of the apparatus main body 4.

In addition, the main probe 1 and the apparatus main body 2 may be connected to each other by wired communication or may be connected to each other by wireless communication. In a case where the main probe 1 and the apparatus main body 2 are connected to each other by wireless communication, and the communication circuit 14 of the main probe 1 and the communication circuit 34 of the subordinate probe 3 are connected to each other by wireless communication, a communication system between the main probe 1 and the apparatus main body 2 and a communication system between the communication circuit 14 of the main probe 1 and the communication circuit 34 of the subordinate probe 3 can be made different from each other. That is, for example, the communication system between the communication circuit 14 of the main probe 1 and the communication circuit 34 of the subordinate probe 3 can be a first wireless system, and the communication system between the main probe 1 and the apparatus main body 2 can be a second wireless system different the first wireless system.

The communication system between the main probe 1 and the apparatus main body 2 and the communication system between the communication circuit 14 of the main probe 1 and the communication circuit 34 of the subordinate probe 3 may be the same wireless system. However, in order to prevent the communication system between the main probe 1 and the apparatus main body 2 from becoming electromagnetic noise for wireless communication between the communication circuit 14 of the main probe 1 and the communication circuit 34 of the subordinate probe 3, it is preferable that the communication system between the communication circuit 14 of the main probe 1 and the communication circuit 34 of the subordinate probe 3 is the first wireless system, and the communication system between the main probe 1 and the apparatus main body 2 is the second wireless system.

In addition, the subordinate probe 3 and the apparatus main body 4 can be connected to each other by wired communication or wireless communication. In a case where the communication circuit 14 of the main probe 1 and the communication circuit 34 of the subordinate probe 3 are connected by wireless communication, and the subordinate probe 3 and the apparatus main body 4 are connected by wireless communication, as the wireless system between the communication circuit 14 of the main probe 1 and the communication circuit 34 of the subordinate probe 3 and the wireless system between the subordinate probe 3 and the apparatus main body 4, the same system may be used, or different systems may be used. However, in order to prevent the communication system between the subordinate probe 3 and the apparatus main body 4 from becoming electromagnetic noise for wireless communication between the communication circuit 14 of the main probe 1 and the communication circuit 34 of the subordinate probe 3, it is preferable that the communication system between the communication circuit 14 of the main probe 1 and the communication circuit 34 of the subordinate probe 3 is the first wireless system, and the communication system between the subordinate probe 3 and the apparatus main body 4 is the second wireless system.

Further, although it has been described that the synchronization signal is directly transmitted and received between the main probe 1 and the subordinate probe 3, the synchronization signal can be indirectly transmitted and received between the main probe 1 and the subordinate probe 3 via the apparatus main body 2 and the apparatus main body 4 by providing the communication circuit 14 in the apparatus main body 2 and providing the communication circuit 34 in the apparatus main body 4.

Further, although it has been described that the synchronization period TC1 is automatically extended by the synchronization period extension unit 15 in a case where the synchronization between the main probe 1 and the subordinate probe 3 has failed, the synchronization period extension unit 15 can also extend the synchronization period TC1 based on the input operation of the user via the input device 25 of the apparatus main body 2 or the input device 45 of the apparatus main body 4. In a case where the probe controller 16 of the main probe 1 determines that the synchronization has failed, the synchronization period extension unit 15 can, for example, display a message asking whether or not to extend the synchronization period TC1 on the monitor 23 of the apparatus main body 2 or the monitor 43 of the apparatus main body 4. The synchronization period extension unit 15 can extend the synchronization period TC1 in a case where an instruction to extend the synchronization period TC1 is input by the user via the input device 25 of the apparatus main body 2 or the input device 45 of the apparatus main body 4.

Further, although it has been described that the synchronization period extension unit 15 extends the synchronization period TC1 in a stepwise manner each time the synchronization between the main probe 1 and the subordinate probe 3 fails, a maximum value of the extension of the synchronization period TC1, for example, 100 milliseconds to 1 second, can be set in advance. As a result, it is possible to prevent the synchronization period TC1 from becoming excessively long, and to extend the synchronization period TC1 to such an extent that the user can smoothly perform the examination.

In addition, the synchronization period extension unit 15 can extend the synchronization period TC1 up to the maximum value of the extension at one time. However, in order not to decrease the frame rate of the ultrasound image more than necessary, it is preferable to extend the synchronization period TC1 in a stepwise manner.

Further, in a case where the synchronization between the main probe 1 and the subordinate probe 3 has failed a predetermined number of times, the probe controller 16 of the main probe 1 or the probe controller 36 of the subordinate probe 3 may display a message indicating a warning on the monitor 23 via the main body controller 24 of the apparatus main body 2 or may display the message on the monitor 43 via the main body controller 44 of the apparatus main body 4. As a result, the user can easily understand that the communication state between the main probe 1 and the subordinate probe 3 has deteriorated and take countermeasures for recovering the communication state.

In addition, the synchronization period extension unit 15 can also set the already extended synchronization period TC1 such that the already extended synchronization period TC1 does not automatically return to an initial value, but returns to the initial value only by the input operation of the user via the input device 25 of the apparatus main body 2 or the input device 45 of the apparatus main body 4. Accordingly, an appropriate length of the synchronization period TC1 can be set according to the communication state between the main probe 1 and the subordinate probe 3 for each examination, and an ultrasound image can be obtained at a stable frame rate.

Further, the synchronization period extension unit 15 may be provided in the subordinate probe 3 instead of the main probe 1, or may be provided in both the main probe 1 and the subordinate probe 3.

In addition, although it has been described that the ultrasound diagnostic apparatus comprises two ultrasound probes of the main probe 1 and the subordinate probe 3, the ultrasound diagnostic apparatus can also comprise a plurality of three or more ultrasound probes. In addition, in this case, the ultrasound diagnostic apparatus can also include a plurality of three or more apparatus main bodies corresponding to the plurality of three or more ultrasound probes.

Although not shown, an example will be described in which the ultrasound diagnostic apparatus comprises three ultrasound probes of the main probe 1, a first subordinate probe, and a second subordinate probe. The first subordinate probe and the second subordinate probe are the same as the subordinate probe 3 shown in FIG. 1. The main probe 1, the first subordinate probe, and the second subordinate probe are communicatively connected to each other.

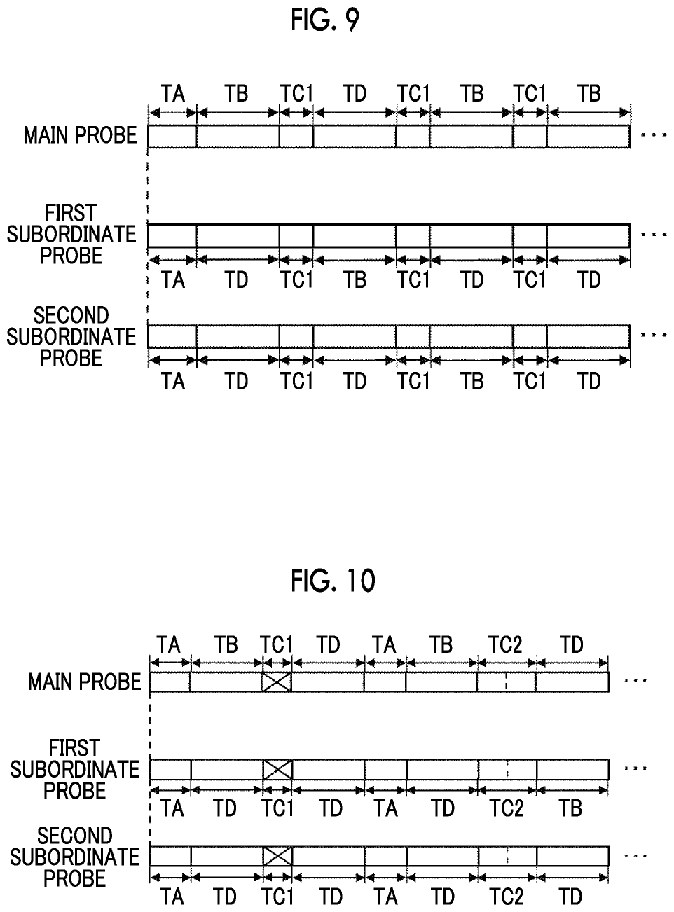

In this case, the main probe 1, the first subordinate probe, and the second subordinate probe can operate according to, for example, a time sequence as shown in FIG. 9. In the time sequence of FIG. 9, the main probe 1 operates in the order of the transmission/reception condition setting period TA, the ultrasonic wave transmission/reception period TB, the synchronization period TC1, the waiting period TD, the synchronization period TC1, the waiting period TD, the synchronization period TC1, and the ultrasonic wave transmission/reception period TB . . . The first subordinate probe operates in the order of the transmission/reception condition setting period TA, the waiting period TD, the synchronization period TC1, the ultrasonic wave transmission/reception period TB, the synchronization period TC1, the waiting period TD, the synchronization period TC1, and the waiting period TD . . . The second subordinate probe operates in the order of the transmission/reception condition setting period TA, the waiting period TD, the synchronization period TC1, the waiting period TD, the synchronization period TC1, the ultrasonic wave transmission/reception period TB, the synchronization period TC1, and the waiting period TD . . . .

In this way, the main probe 1, the first subordinate probe, and the second subordinate probe operate such that only one of the main probe 1, the first subordinate probe, and the second subordinate probe is set to the ultrasonic wave transmission/reception period TB.

For example, as shown in FIG. 10, in a case where the synchronization between the main probe 1, the first subordinate probe, and the second subordinate probe has failed in the first synchronization period TC1, the synchronization period extension unit 15 of the main probe 1 extends the synchronization period TC1 to set the synchronization period TC2. Thereafter, the main probe 1, the first subordinate probe, and the second subordinate probe all enter the waiting period TD, and the operations of the main probe 1, the first subordinate probe, and the second subordinate probe are restarted from the transmission/reception condition setting period TA. After the operations of the main probe 1, the first subordinate probe, and the second subordinate probe are restarted, the extended synchronization period TC2 is used.

As described above, even in a case where the ultrasound diagnostic apparatus comprises a plurality of three or more ultrasound probes, in the same manner as in a case where the ultrasound diagnostic apparatus comprises two ultrasound probes of the main probe 1 and the subordinate probe 3, in a case where the synchronization between the plurality of ultrasound probes has failed, since the synchronization period extension unit 15 extends the synchronization period TC1, the plurality of ultrasound probes can be more reliably synchronized with each other, and the user can smoothly perform the examination on the subject.

In addition, although it has been described that the ultrasound diagnostic apparatus may comprise a plurality of apparatus main bodies corresponding to a plurality of ultrasound probes, the ultrasound diagnostic apparatus can also comprise only one apparatus main body.

In addition, the apparatus main bodies 2 and 5 may be configured of an easily portable device such as a so-called tablet-type computer and a so-called smartphone, or may be configured of a so-called stationary device. In a case where the ultrasound diagnostic apparatus comprises only one apparatus main body, it is preferable that the ultrasound diagnostic apparatus is configured of a stationary device capable of comprising a monitor larger than that of an easily portable device such as a tablet-type computer and a smartphone such that the user can easily check the monitor of the apparatus main body.

In addition, for example, in a case where the apparatus main body 2 connected to the main probe 1 and the subordinate probe 3 are communicatively connected, the image processing unit 21 of the apparatus main body 2 can generate a three-dimensional ultrasound image based on the image data generated by the main probe 1 and the image data generated by the subordinate probe 3. The generated three-dimensional ultrasound image is displayed on the monitor 23 after being subjected to various types of processing by the display controller 22. In addition, in a case where the apparatus main body 4 connected to the subordinate probe 3 and the main probe 1 are communicatively connected, the image processing unit 41 of the apparatus main body 4 can generate a three-dimensional ultrasound image based on the image data generated by the main probe 1 and the image data generated by the subordinate probe 3. The generated three-dimensional ultrasound image is displayed on the monitor 43 after being subjected to various types of processing by the display controller 42.

Similarly, in a case where the ultrasound diagnostic apparatus comprises a plurality of ultrasound probes and a plurality of apparatus main bodies, at least one apparatus main body among the plurality of apparatus main bodies can generate a three-dimensional ultrasound image based on the image data transmitted from each of the plurality of ultrasound probes. The three-dimensional ultrasound image generated in this way is displayed on a monitor of at least one apparatus main body among the plurality of apparatus main bodies.

Second Embodiment

Although it has been described that the ultrasound image is displayed on the monitor 23 of the apparatus main body 2 based on the image data generated by the main probe 1, and the ultrasound image is displayed on the monitor 43 of the apparatus main body 4 based on the image data generated by the subordinate probe 3, the ultrasound image may be displayed on a display device having a relatively large monitor such that the user can easily check the ultrasound image.

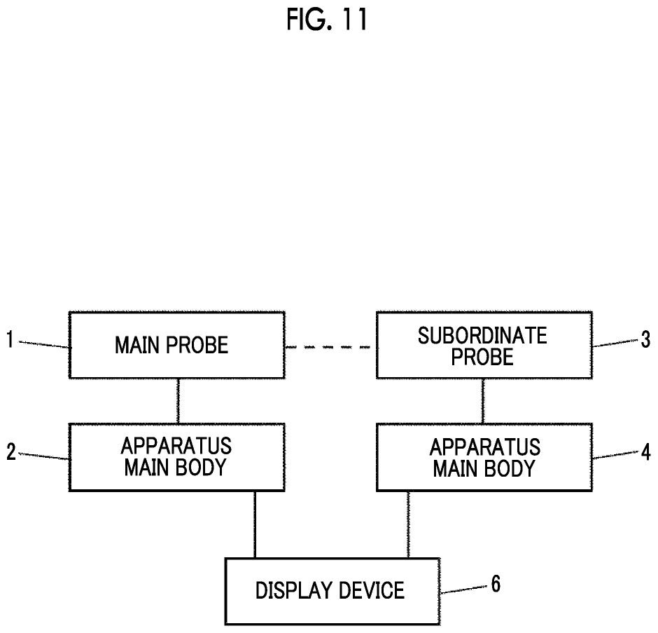

FIG. 11 shows an ultrasound diagnostic apparatus according to the second embodiment. The ultrasound diagnostic apparatus is configured such that a display device 6 is added to the ultrasound diagnostic apparatus according to the first embodiment shown in FIG. 1. In the ultrasound diagnostic apparatus according to the second embodiment, the display device 6 is connected to the apparatus main body 2 and the apparatus main body 4.

The display device 6 is configured of a monitor relatively larger than the monitor 23 of the apparatus main body 2 and the monitor 43 of the apparatus main body 4. Both the ultrasound image generated by the apparatus main body 2 and the ultrasound image generated by the apparatus main body 4 are transmitted to the display device 6, and these two ultrasound images are simultaneously displayed on the display device 6.

As a result, the user can easily check the two ultrasound images simultaneously displayed on the display device 6 and can more smoothly perform the examination on the subject.

In addition, similarly to the ultrasound diagnostic apparatus according to the first embodiment, the ultrasound diagnostic apparatus according to the second embodiment can comprise a plurality of three or more ultrasound probes and a plurality of three or more apparatus main bodies corresponding to the plurality of three or more ultrasound probes. Although it may be difficult for the user to check all monitors of the plurality of apparatus main bodies in order to check a plurality of obtained ultrasound images, the user can easily check the plurality of ultrasound images that are simultaneously displayed by checking one display device 6.

In addition, at least one apparatus main body among the plurality of apparatus main bodies in the second embodiment can generate a three-dimensional ultrasound image based on the image data transmitted from each of the plurality of ultrasound probes in the same manner as in the first embodiment. In this case, the display device 6 can display the three-dimensional ultrasound image. The user can easily and more precisely check the three-dimensional ultrasound image by checking the display device 6 configured of a monitor relatively larger than the monitors of the plurality of apparatus main bodies.

### Explanation of References

1: main probe

2, 4: apparatus main body

3: subordinate probe

6: display device

11, 31: transducer array

12, 32: transmission/reception circuit

13, 33: image data generation unit

14, 34: communication circuit

15: synchronization period extension unit

16, 36: probe controller

17, 26, 37, 46: processor

21, 41: image processing unit

22, 42: display controller

23, 43: monitor

24, 44: main body controller

25, 45: input device

51: pulser

52: amplification unit

53: AD conversion unit

54: beam former

55: signal processing unit

56: DSC

TA: transmission/reception condition setting period

TB: ultrasonic wave transmission/reception period

TC1, TC2, TC3: synchronization period

TD: waiting period

TE: extension period

What is claimed is:

1. An ultrasound diagnostic apparatus comprising:
a plurality of ultrasound probes each transmitting and receiving ultrasonic waves,
wherein the plurality of ultrasound probes have communication circuits configured to notify of an operating state regarding transmission and reception of the ultrasonic waves by transmitting synchronization signals to each other,
each of the plurality of ultrasound probes is configured to operate according to a time sequence including at least an ultrasonic wave transmission/reception period for transmitting and receiving the ultrasonic waves, a waiting period for stopping transmission and reception of the ultrasonic waves, and a synchronization period for synchronization between the ultrasound probes,
in a case where any one of the plurality of ultrasound probes is configured to be in the ultrasonic wave transmission/reception period, the other ultrasound probe is configured to be in the waiting period,
in a case where any one of the plurality of ultrasound probes is configured to be in the synchronization period, the other ultrasound probe is also configured to be in the synchronization period, and
in a case where the plurality of ultrasound probes are configured to fail to synchronize with each other in the synchronization period, the synchronization period is extended and after the plurality of ultrasound probes becomes a state capable of synchronizing with each other, any one of the plurality of ultrasound probes is configured to be in the ultrasonic wave transmission/reception period.

2. The ultrasound diagnostic apparatus according to claim 1,
wherein in a case where the synchronization period is extended, the synchronization period is returned to a length before the extension only in a case where the user operates.

3. The ultrasound diagnostic apparatus according to claim 2,
wherein one ultrasound probe of the plurality of ultrasound probes is configured to operate as a main probe, and the remaining ultrasound probe is configured to operate as a subordinate probe, and
the main probe is configured to extend the synchronization period in a case where the communication circuit is not capable of detecting the synchronization signal transmitted from the subordinate probe in the synchronization period.

4. The ultrasound diagnostic apparatus according to claim 3,
wherein the main probe is configured to automatically extend the synchronization period in a case where the communication circuit is not capable of detecting the synchronization signal transmitted from the subordinate probe in the synchronization period.

5. The ultrasound diagnostic apparatus according to claim 3,
wherein the main probe is configured to extend the synchronization period based on the input operation by the user.

6. The ultrasound diagnostic apparatus according to claim 1,
wherein a maximum value of the extension of the synchronization period is defined.

7. The ultrasound diagnostic apparatus according to claim 2,
wherein a maximum value of the extension of the synchronization period is defined.

8. The ultrasound diagnostic apparatus according to claim 3,
wherein a maximum value of the extension of the synchronization period is defined.

9. The ultrasound diagnostic apparatus according to claim 1,
wherein the synchronization period is extended in a stepwise manner.

10. The ultrasound diagnostic apparatus according to claim 2,
wherein the synchronization period is extended in a stepwise manner.

11. The ultrasound diagnostic apparatus according to claim 3,
wherein the synchronization period is extended in a stepwise manner.

12. The ultrasound diagnostic apparatus according to claim 1, further comprising a plurality of apparatus main bodies that correspond to the plurality of ultrasound probes and are connected to the plurality of ultrasound probes, respectively,
wherein each of the plurality of ultrasound probes is configured to
acquire image data by transmitting and receiving the ultrasonic waves in the ultrasonic wave transmission/reception period and
transmit the image data to the corresponding apparatus main body.

13. The ultrasound diagnostic apparatus according to claim 2, further comprising a plurality of apparatus main bodies that correspond to the plurality of ultrasound probes and are connected to the plurality of ultrasound probes, respectively,
wherein each of the plurality of ultrasound probes is configured to
acquire image data by transmitting and receiving the ultrasonic waves in the ultrasonic wave transmission/reception period and
transmit the image data to the corresponding apparatus main body.

14. The ultrasound diagnostic apparatus according to claim 12,
wherein each of the plurality of ultrasound probes is configured to
transmit the synchronization signal to the other ultrasound probe by wireless communication, and
transmit the image data to the corresponding apparatus main body by wired communication.

15. The ultrasound diagnostic apparatus according to claim 12,
wherein each of the plurality of ultrasound probes is configured to
transmit the synchronization signal to the other ultrasound probe by a first wireless system, and
transmit the image data to the corresponding apparatus main body by a second wireless system.

16. The ultrasound diagnostic apparatus according to claim 12,
wherein the plurality of ultrasound probes are configured to transmit the synchronization signal to each other via the plurality of apparatus main bodies.

17. The ultrasound diagnostic apparatus according to claim 12, further comprising one display device connected to the plurality of apparatus main bodies,
wherein a plurality of ultrasound images are simultaneously displayed on the display device based on a plurality of image data transmitted from the plurality of ultrasound probes to the plurality of apparatus main bodies.

18. The ultrasound diagnostic apparatus according to claim 17,
wherein at least one apparatus main body among the plurality of apparatus main bodies is configured to generate a three-dimensional ultrasound image based on the image data transmitted from each of the plurality of ultrasound probes, and
the three-dimensional ultrasound image is displayed on the display device.

19. The ultrasound diagnostic apparatus according to claim 12, wherein each of the plurality of ultrasound probes includes
a transducer array,
a transmission/reception circuit configured to transmit ultrasonic waves from the transducer array and generate a sound ray signal based on a reception signal acquired by the transducer array, and
a processor configured to generate the image data based on the sound ray signal generated by the transmission/reception circuit.

20. A control method of an ultrasound diagnostic apparatus including a plurality of ultrasound probes each transmitting and receiving ultrasonic waves, the control method comprising:
by the plurality of ultrasound probes, notifying of an operating state regarding transmission and reception of the ultrasonic waves by transmitting synchronization signals to each other,
wherein each of the plurality of ultrasound probes operates according to a time sequence including an ultrasonic wave transmission/reception period for transmitting and receiving the ultrasonic waves, a waiting period during which transmission and reception of the ultrasonic waves are not performed, and a synchronization period for synchronization between the ultrasound probes,
in a case where any one of the plurality of ultrasound probes is in the ultrasonic wave transmission/reception period, the other ultrasound probe is in the waiting period,
in a case where any one of the plurality of ultrasound probes is in the synchronization period, the other ultrasound probe is also in the synchronization period, and
in a case where the plurality of ultrasound probes fail to synchronize with each other in the synchronization period, the synchronization period is extended and after the plurality of ultrasound probes becomes a state capable of synchronizing with each other, any one of the plurality of ultrasound probes is in the ultrasonic wave transmission/reception period.
